(19) United States
(12) Patent Application Publication
(10) Pub. No.: US 2005/0165312 A1
(43) Pub. Date: Jul. 28, 2005

(54) Acoustic window for ultrasound probes

(76) Inventors: Heather Beck Knowles, Devens, MA (US); Jacquelyn M.B. Byron, Arlington, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/040,055

(22) Filed: Jan. 21, 2005

Related U.S. Application Data
(60) Provisional application No. 60/539,302, filed on Jan. 26, 2004.

(51) Int. Cl. A61B 8/14

(52) U.S. Cl. 600/459

(57) ABSTRACT

Acoustic window for an ultrasound probe including a layer of PEBAX™ having a surface adapted to face a transducer array and an opposed surface adapted to face the object being examined and optionally impervious polymer layers arranged on one or both surfaces of the PEBAX™ layer. An outer polymer layer on the object-facing surface, when present, protects the PEBAX™ layer from chemicals while the inner polymer layer on the transducer array-facing surface, when present, is bonded to the transducer array of the probe. The polymer layers may each be made from an impervious polymer which has a negligible acoustic impact including, for example, polyethylene, Mylar™ and Kapton™. A method for manufacturing the acoustic window and an ultrasound probe including the same are also disclosed.

### CROSS REFERENCE TO RELATED CASES

Applicants claim the benefit of Provisional Application Ser. No. 60/539,302, filed 26 Jan. 2004.

### FIELD OF THE INVENTION

The present invention relates generally to ultrasound probes and more particularly to an acoustic window for ultrasound probes and a method for manufacturing the same.

### BACKGROUND OF THE INVENTION

Ultrasound probes are commonly used for imaging internal body parts. Typically, an ultrasound probe includes an outer hard plastic body, usually referred to as a housing for transesophageal echocardiographic (TEE) probes and as a nose for transthoracic or intracavity probes, an acoustic window fixed to the housing/nose and a transducer array arranged in the housing/nose. The transducer array is sometimes referred to in the art as an acoustic stack assembly and usually includes a layer of piezoelectric material, one or more acoustic matching layers adjacent the active surface of the piezoelectric material and a backing layer on the reverse side of the piezoelectric material.

During use of all of these possible products, an outer surface of the acoustic window is exposed to the surrounding environment and thus the acoustic window serves as the interface between the transducer array and the surrounding environment. Ultrasonic waves generated by the transducer array pass through the acoustic window in their path toward and from the body parts being imaged. The acoustic window also contacts the patient to ensure optimal acoustic conditions.

Various factors are considered when selecting materials from which to construct the acoustic window of an ultrasound probe. It is desired that the acoustic window is formed from a material that has an acoustic impedance which matches, or at least closely approximates, that of the human body part being imaged, such as the tissue of the human body. Acoustic impedance is based on the elasticity, mass density and speed of sound of the material. Additional characteristics of the material include acceptable mechanical and electrical performance, biocompatibility, chemical resistance, low attenuation and stability to ultraviolet rays.

One particular material that has excellent mechanical and electrical performance, the desired impedance and velocity characteristics of the human body as well as low attenuation is a particular polymer block amide called PEBAX™ (sold by Autochem). However, an unfortunate property of PEBAX™ is its acute susceptibility to solvents, particularly isopropyl alcohol, which is a disinfectant commonly used to clean ultrasound probes after use. Disinfection of ultrasound probes should be performed after each use of the probe, especially for transesophageal echocardiographic (TEE) probes which are inserted into a patient's throat during the imaging procedure. Disinfection is also required for intracavity probes, which are usually highly contaminated, and for intraoperative probes, which must be high-level disinfected and used with a sterile sheath. In view of this susceptibility, PEBAX™ was not considered for use in an acoustic window of a reusable ultrasound probe.

### OBJECTS AND SUMMARY OF THE INVENTION

It is an object of the present invention to provide a new and improved acoustic window for use in ultrasound probes, ultrasound probes including the same and a method for manufacturing the same.

It is still another object of the present invention to provide a new and improved acoustic window including PEBAX™ which overcomes the problem which prevented the use of PEBAX™ in prior art acoustic windows, and an ultrasound probe including the same.

It is yet another object of the present invention to provide a new and improved method for manufacturing an acoustic window including PEBAX™ for use in an ultrasound probe and an acoustic window manufactured thereby.

In order to achieve these objects and others, an acoustic window for an ultrasound probe in accordance with the invention includes a PEBAX™ layer having a first surface adapted to face a transducer array and an opposed surfaces adapted to face outward toward the object being examined, a first layer of an impervious polymer arranged on the first surface of the PEBAX™ layer and a second layer of an impervious polymer arranged on the second surface of the PEBAX™ layer. The PEBAX™ layer is thus sandwiched between the polymer layers. The polymer layers may be bonded to the PEBAX™ layer, e.g., by using heat and/or a primer, with the PEBAX™ itself acting as an adhesive, or by providing an adhesive between the polymer layers and the PEBAX™ layer.

The polymer layers may each be made from any type of impervious polymer which preferably has a negligible acoustic impact including, but not limited to, polyethylene, Mylar™ and Kapton™. A different polymer can be used for each polymer layer if desired or the same polymer can be used for both layers.

An alternative construction of the acoustic window includes a layer of PEBAX™ having opposed surfaces and only a layer of an impervious polymer arranged on an upper surface of the PEBAX™ layer which is designed to be exposed to the ambient atmosphere, i.e., come into contact with the patient. In this case, the PEBAX™ layer is attached directly to the upper surface of the transducer array, possibly by heat, pressure, primers and/or adhesives. Also, the polymer layer would preferably be sealed with an impervious seal to the housing of the probe into which the transducer assembly is installed in order to prevent ingress of solvent into the PEBAX or the probe.

Another alternative construction of the acoustic window would be to provide only a polymer layer between the PEBAX™ layer and the transducer array in which case the upper surface of the PEBAX™ layer is exposed. In yet another alternative construction, the acoustic window could consist of only the PEBAX™ layer, without any covering polymer layers. In both of these constructions, care would have to be taken to limit solvent exposure.

A method for manufacturing an acoustic window in accordance with the invention entails providing a layer of PEBAX™ and sandwiching the PEBAX™ layer between a first impervious polymer layer and a second impervious polymer layer. The polymer layers are preferably bonded to the PEBAX™ layer to prevent separation therefrom.

An ultrasound probe in accordance with the invention includes a housing defining a cavity extending inward from a peripheral surface, a transducer array arranged in the cavity of the housing to produce ultrasound beams, and an acoustic window as described above attached or bonded directly to the transducer array. The transducer array may include independently-addressable transducer elements such that ultrasound beams in various planes and volumes can be generated thereby. The housing may be in the form of a housing for a transesophageal echocardiographic ultrasound probe or transnasal probe, or a nose for a transthoracic, intracavity or intraoperative probe. Thus, as used herein, a “housing” of an ultrasound probe may be either a housing per se or a nose depending on the type of probe.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a cross-sectional view of a portion of a first embodiment of a generic ultrasound probe taken through the transducer assembly.

FIG. 2 is an enlarged view of the section designated A in FIG. 1.

FIG. 3 is a cross-sectional view of a portion of a second embodiment of a generic ultrasound probe in accordance with the invention taken through the transducer assembly.

FIG. 4 is a cross-sectional view of a portion of a third embodiment of a generic ultrasound probe in accordance with the invention taken through the transducer assembly.

FIG. 5 is a cross-sectional view of a portion of a fourth embodiment of a generic ultrasound probe in accordance with the invention taken through the transducer assembly.

FIG. 6 is a cross-sectional view of a portion of a fifth embodiment of a generic ultrasound probe in accordance with the invention taken through the transducer assembly.

FIG. 7 is a cross-sectional view of a portion of a sixth embodiment of a generic ultrasound probe in accordance with the invention taken through the transducer assembly.

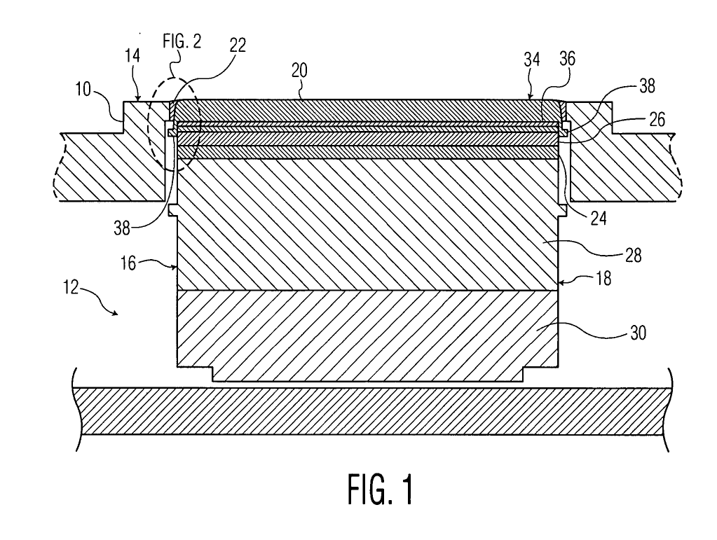

### DETAILED DESCRIPTION OF THE INVENTION

Referring to the accompanying drawings wherein like reference numerals refer to the same or similar elements, an ultrasound probe in accordance with the invention includes an outer hard plastic body 10, referred to herein as a housing, defining a cavity 12 extending inward from an opening in a peripheral surface 14 and a transducer assembly 16 arranged in the cavity 12. Housing 10 can be shaped in the form of any type of transthoracic, intracavity, TEE, transnasal probe or any other type of ultrasound probe. Housing 10 and the transducer assembly 16 in accordance with the invention can also be used in any imaging device in the medical field.

Transducer assembly 16 includes a transducer array 18 and an acoustic window 20 fixed, attached or bonded directly to the transducer array 18 to thereby form a transducer assembly 16 with an integral acoustic window 20. The direct fixing or attachment of the acoustic window 20 to the transducer array 18 may be accomplished through the use of an adhesive. The transducer array 18 includes a layer of piezoelectric material 24, one or more acoustic matching layers 26 adjacent the active surface of the piezoelectric material 24 and a backing layer 28 on the reverse side of the piezoelectric material 24.

To prevent fluids which come into contact with the acoustic window 20 from entering into the interior of the housing 10 during use of the ultrasound probe, a seal 22 is arranged between the housing 10 and the acoustic window 20. The seal 22 is made of a flexible material and should be sufficiently impermeable to fluids and materials such as those used during an ultrasound examination which are known to those skilled in the art.

The flexible seal 22 may be matched with the durometer of the acoustic window 20 and will thus conform to the shape of the acoustic window 20 if the acoustic window 20 is impacted.

Transducer array 18 includes a layer of piezoelectric material 24, an acoustic matching section 26 adjacent the active surface of the piezoelectric layer 24 and a backing layer 28 on the opposite side of the piezoelectric layer 24 from the acoustic matching section 26. The acoustic matching section 26 may contain one or more acoustic matching layers. The acoustic window 20 is attached to the upper surface of the acoustic matching section 26. Although a heatsink 30 is shown under the backing layer 28, this is an optional feature and is used only in certain types of ultrasound probes.

The transducer array 18 is preferably constructed as a matrix array for which it is not necessary to provide lateral focus through the use of a lens. The transducer elements in the piezoelectric layer 24 are thus independently addressable and are connected to an integrated circuit which is connected in turn to a circuit board. The transducer elements may be segmented into (or designated as) transmit sub-arrays and receive sub-arrays. Each transmit sub-array may be connected to a respective intra-group transmit pre-processor which is connected to a respective transmit beamformer channel. Each receive sub-array may be connected to a respective intra-group receive pre-processor which is connected to a respective receive beamformer channel. Control of the sub-arrays is obtained by a control processor in a manner known in the art, for example, as disclosed in U.S. Pat. No. 6,572,547 incorporated by reference herein.

A matrix array will therefore be considered as a type of transducer array which is capable of generating multi-planar images on an object by appropriate electronic control of transducer elements of the transducer array.

Alternatively, a conventional two-dimensional array in which the transducer elements themselves are curved to provide the focus could be used in combination with the acoustic window 20. Furthermore, in another alternative use, the acoustic window 20 could be bonded on top of a conventional lens material to form a window or standoff.

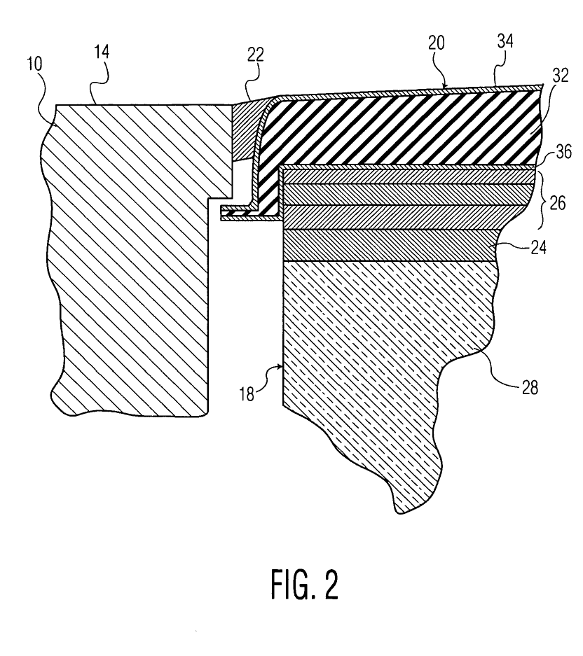

Referring now to FIG. 2, in accordance with the invention, the acoustic window 20 includes an elastomer layer 32 sandwiched between upper and lower polymer layers 34, 36, respectively, with the upper polymer layer 34 defining an outer, exposed surface of the transducer assembly 16. The lower polymer layer 36 is attached directly to the upper surface of the acoustic matching section 26 of the transducer array 18 without any gap therebetween.

The elastomer layer 32 is made of PEBAX™ which is sandwiched between the two polymer layers 34,36, e.g.;, thin films of polymer, and the polymer layers 34, 36 and PEBAX™ layer 32 are bonded to one another. The polymer layers 34,36 will therefore not separate from the PEBAX™ layer 32 during mechanical stress or environmental cycling.

Bonding of the polymer layers 34,36 to the PEBAX™ layer 32 can be accomplished in several ways. For example, the PEBAX™ layer 32 can be primed and/or heated, such that it acts as an adhesive to cause the polymer layers 34,36 to be bonded thereto. In addition to or instead of priming the PEBAX™ layer 32, it is possible to provide adhesive between each polymer layer 34,36 and the PEBAX™ layer 32.

Bonding of materials directly to PEBAX™ is often problematic. Thus, in the invention, by providing the lower polymer layer 36, the transducer array 18 can be bonded more easily to the lower polymer layer 36 than to the PEBAX™ layer 32. On the other hand, providing the upper polymer layer 34 protects the PEBAX™ layer 32 from scratches and other types of mechanical damage and also creates a barrier that eliminates chemical susceptibility.

PEBAX™ is a moldable material and therefore lends itself nicely to the production of acoustic windows having various shapes and sizes. PEBAX™ can also be blended with polyethylene or other materials to tailor its properties. It is available in a range of durometers, several of which are appropriate for use in an acoustic window.

The polymer layers 34, 36 may each be made from any type of impervious polymer which preferably has a negligible acoustic impact including, but not limited to, polyethylene, Mylar™ and Kapton™. A different polymer can be used for each polymer layer 34, 36 if desired or the same polymer can be used for both polymer layers 34,36.

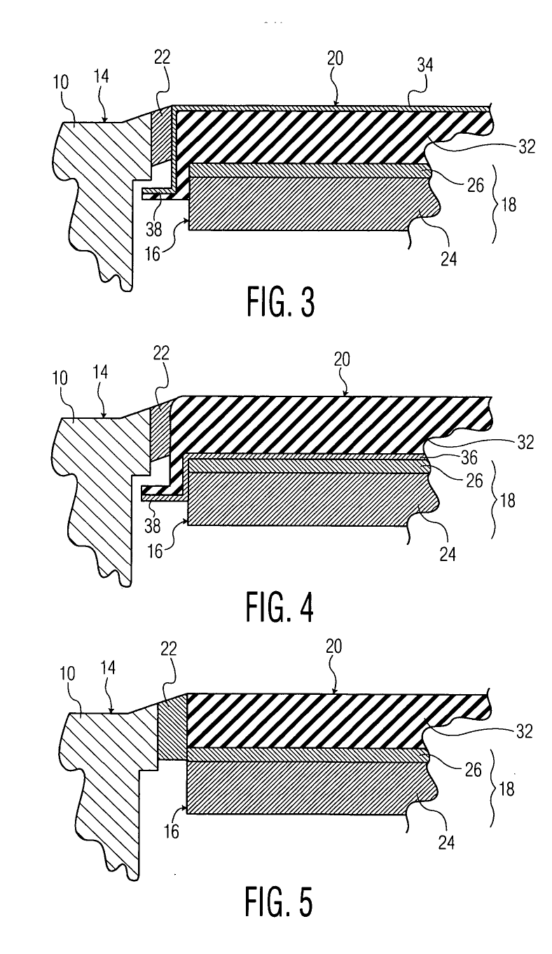

Although the embodiment of the acoustic window 20 shown in FIG. 2 includes both an upper polymer layer 34 and a lower polymer layer 36, it is possible to construct an acoustic window without the lower polymer layer 36. In this embodiment, shown in FIG. 3, the PEBAX™ layer 32 is bonded directly to or formed directly on the upper surface of the transducer array 18, i.e., the upper surface of the acoustic matching section 26 of the transducer array 18.

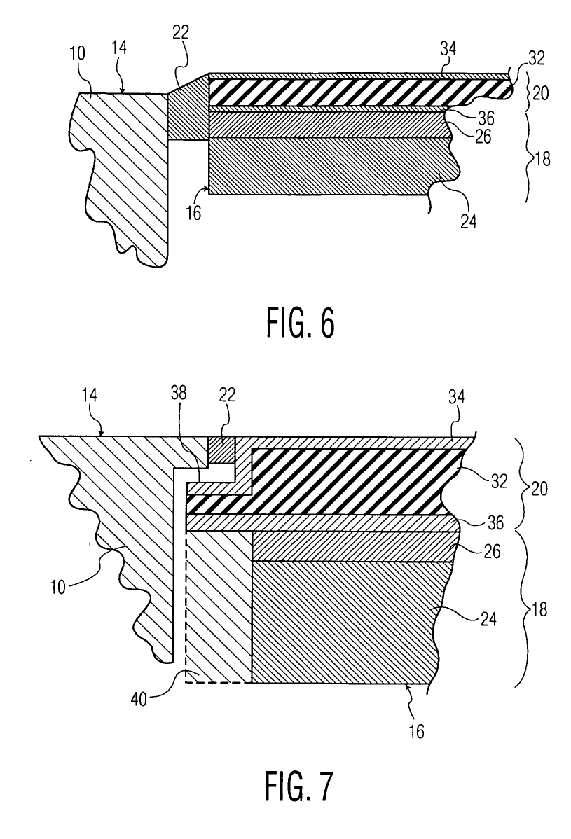

The acoustic window 20 may have a larger cross-sectional area than the acoustic matching section 26 so that a portion of the acoustic window 20 is situated alongside the acoustic matching 26. The lower polymer layer 36 is therefore bonded to the lateral edge of the acoustic matching section 26 (see FIG. 2) or may have a cross-sectional area which is substantially the same as the acoustic matching section 26 (see FIGS. 5 and 6). The acoustic window 20 has an extended section 38 alongside an upper portion of the transducer array 18 which serves to create a convoluted path (in combination with the peripheral surface of the cavity of the housing 10 into which the transducer assembly 16 is placed) to prevent fluids from entering into the interior of the housing 10. This improves the electrical safety of the probe. Further, the presence of the upper polymer layer 34 provides an easy bonding of the acoustic window 20 to the seal 22.

By covering at least the portion of the PEBAX™ layer 32 which would otherwise be exposed to the surrounding environment with the upper polymer layer 34, the outer, exposed surface of the acoustic window 20 is defined by the upper polymer layer 34 and therefore disinfectants such as isopropyl alcohol can come into contact with the upper polymer layer 34 but do not come into contact with PEBAX™ layer 32. This avoids the problems which arise when PEBAX™ layer 32 comes into contact with such disinfectants.

The acoustic window 20 described above can be used in various types of ultrasound probes having transducer arrays. For example, the acoustic window can be formed in a transesophageal echocardiographic (TEE) ultrasound probe, transnasal ultrasound probe, a transnasal echocardiographic ultrasound probe, an intra-operative ultrasound probe or an intracavity ultrasound probe.

It is also envisioned that the acoustic window 20 is formed without the upper polymer layer 36, i.e., with only a single lower polymer layer 36 between the transducer array 18 and the acoustic window 20 (as shown in FIG. 4), or without both the upper and lower polymer layers 34,36 (as shown in FIG. 5) with the PEBAX™ layer 32 being formed directly on the transducer array 18 as discussed above. In this case, since the PEBAX™ layer 32 is exposed to the surrounding environment, the use of disinfectants which are not compatible with PEBAX™ would be prohibited. These designs could also be utilized for a single-use device.

FIG. 6 shows an embodiment wherein the acoustic window 20 includes the PEBAX™ layer 32 and the upper and lower polymer layers 34,36. In this embodiment, as well as in the embodiment shown in FIG. 5, the acoustic window 20 does not extend beyond the lateral edges of the transducer array 18. Thus, the dimensions of the transducer array 18 and the acoustic window 20 are substantially the same. The seal 22 thus engages the acoustic window 20 and a part of the transducer array 18.

FIG. 7 shows another embodiment wherein the acoustic window 20 includes the PEBAX™ layer 32 and the upper and lower polymer layers 34,36. In this embodiment, the acoustic window 20 does not have an extended portion 38 alongside the transducer array 18 and thus is entirely above the transducer array 18. That is, as shown in FIGS. 2-4, a portion of the acoustic window 20 is alongside the acoustic matching section 26 of the transducer array 18. By appropriate construction of the housing 10, it is possible to create a convoluted path between the housing 10 and the acoustic window 20 to prevent fluids from entering into interior of the housing. To support the portion of the acoustic window 20 extending laterally beyond the transducer array 18, an optional support 40 (shown in dotted lines) may be provided.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. An acoustic window for an ultrasound probe, comprising:
a layer of PEBAX™ having a first surface adapted to face a transducer array and an opposed second surface adapted to face an object being examined;
an optional first layer of an impervious polymer arranged on said first surface of said PEBAX™ layer; and
an optional second layer of an impervious polymer arranged on said second surface of said PEBAX™ layer such that when said first and second polymer layers are present, said PEBAX™ layer is sandwiched between said first and second impervious polymer layers.

2. The acoustic window of claim 1, wherein the acoustic window includes said first and second polymer layers and said first and second polymer layers are bonded to said PEBAX™ layer.

3. The acoustic window of claim 2, further comprising an adhesive interposed between said first and second polymer layers and said PEBAX™ layer for bonding said first and second polymer layers to said PEBAX™ layer.

4. The acoustic window of claim 1, wherein said first and second polymer layers are each made from polyethylene, Mylar™ or Kapton™.

5. The acoustic window of claim 1, wherein the acoustic window includes said first and second polymer layers and said first and second polymer layers are made from a different impervious polymer.

6. The acoustic window of claim 1, wherein the acoustic window includes only said first polymer layer and not said second polymer layer.

7. The acoustic window of claim 1, wherein the acoustic window includes only said second polymer layer and not said first polymer layer.

8. The acoustic window of claim 1, wherein the acoustic window consists of only said PEBAX™ layer.

9. A method for manufacturing an acoustic window, comprising the steps of:
providing a PEBAX™ layer; and
sandwiching the PEBAX™ layer between a first layer of an impervious polymer and a second layer of an impervious polymer.

10. The method of claim 9, further comprising the step of bonding the first and second polymer layers directly to the PEBAX™ layer.

11. The method of claim 9, wherein the step of bonding the first and second polymer layers to the PEBAX™ layer comprises the step of at least one of priming and heating the PEBAX™ layer such that it acts as an adhesive.

12. The method of claim 9, wherein the step of bonding the first and second polymer layers to the PEBAX™ layer comprises the step of arranging an adhesive between each of the first and second polymer layers and the PEBAX™ layer.

13. An ultrasound probe, comprising:
a housing defining a cavity extending inward from an opening in a peripheral surface;
a transducer array arranged in said cavity of said housing to produce ultrasound beams, said transducer array comprising a plurality of independently-addressable transducer elements such that ultrasound beams in various planes and volumes are generateable by said transducer elements; and
an acoustic window attached to said transducer array, said acoustic window comprising a layer of PEBAX™ having a first surface arranged to face said transducer array and an opposed second surface arranged to face outward, an optional first layer of an impervious polymer arranged on said first surface of said PEBAX™ layer and an optional second layer of an impervious polymer arranged on said second surface of said PEBAX™ layer such that when said first and second polymer layers are present, said PEBAX™ layer is sandwiched between said first and second polymer layers.

14. The ultrasound probe of claim 13, wherein said housing is in the form of a housing of a transesophageal echocardiographic probe, a housing of a transnasal probe, a nose of a transthoracic probe, a nose of an intracavity probe or a nose of an intraoperative probe.

15. The ultrasound probe of claim 13, further comprising a flexible seal interposed between said acoustic window and said housing to seal said cavity.

16. The ultrasound probe of claim 13, wherein said acoustic window includes only said first polymer layer and not said second polymer layer.

17. The ultrasound probe of claim 13, wherein said acoustic window includes only said second polymer layer and not said first polymer layer.

18. The ultrasound probe of claim 13, wherein the acoustic window consists of only said PEBAX™ layer.

19. The ultrasound probe of claim 13, wherein said acoustic window includes said first and second polymer layers and said first and second polymer layers are each made from polyethylene, Mylar™ or Kapton™.

20. The ultrasound probe of claim 13, wherein said acoustic window includes said first and second polymer layers and said first and second polymer layers are made from a different impervious polymer.
